(19) United States
(12) Patent Application Publication
(10) Pub. No.: US 2023/0085578 A1
(43) Pub. Date: Mar. 16, 2023

(54) SYSTEMS AND METHODS FOR TRIPLE-PARAMETRIC OPTICAL MAPPING

(71) Applicant: The George Washington University, Washington, DC (US)

(72) Inventors: Sharon Ann GEORGE, Washington, DC (US); Igor R. EFIMOV, Washington, DC (US)

(21) Appl. No.: 17/901,979

(22) Filed: Sep. 2, 2022

Related U.S. Application Data
(60) Provisional application No. 63/240,242, filed on Sep. 2, 2021.

(51) Int. Cl. A61B 5/00

(52) U.S. Cl. CPC A61B 5/0066; A61B 5/0077; A61B 2018/00351

(57) ABSTRACT

Systems and methods are disclosed for an optical mapping device. The device emits different wavelengths of light from a plurality of light sources to a cardiac tissue and passes the light through a lens, a first filter cube in the path of the light with a first light filter, a second light filter, and a third light filter. Light passing through the filters is recorded by three cameras that each record an indicator of cardiac physiology, which are mapped simultaneously by the device.

### CROSS-REFERENCE TO RELATED APPLICATIONS

This application claims the benefit of U.S. Provisional App. No. 63/240,242, filed Sep. 2, 2021, the entire contents of which are incorporated herein by reference.

### STATEMENT REGARDING FEDERALLY FUNDED RESEARCH

This invention was made with government support under Grant No. R44 HL139248 from the National Institutes of Health. The government has certain rights in the invention.

### FIELD OF THE INVENTION

The present invention is directed to devices and methods for optically mapping cardiac physiology.

### BACKGROUND OF THE INVENTION

Cardiac physiology is profoundly complex, and the careful coordination of numerous cell signaling pathways govern every single heartbeat. These physiological processes that vary beat to beat and long term are usually sub-divided into metabolic, electrical, and mechanical components governed by metabolism-excitation-contraction coupling (MECC). Electrical activity in the heart includes a sequence of opening and closing of ion channels and pumps which cause cardiomyocyte depolarization and repolarization. The resulting changes in the transmembrane potential (Vu) in the cardiomyocytes are recorded as action potentials using electrical or optical methods. The electrical excitation of the heart serves as the trigger of a mechanical contraction. Excitation-contraction coupling or the translation of electrical excitation to mechanical contraction is controlled by cytosolic calcium (Ca²⁺) ion concentration, which increases following electrical excitation and Ca²⁺ ion binds to a contractile protein in the cardiomyocyte, triggering contraction. Both the electrical and mechanical processes of the heart require energy, which is provided in the form of ATP generated by metabolic processes in the mitochondria. Thus, all three components of cardiac function are intertwined into MECC. As such, studying this complex MECC phenomenon requires the ability to simultaneously assess these three facets of cardiac function.

### SUMMARY OF THE INVENTION

The present technology involves several aspects that incorporate improvements over preexisting devices. Exemplarily, the technology includes optical mapping system comprising a plurality of light sources emitting different wavelengths of light to cardiac tissue. There is a first lens situated in a path of the light, a first filter cube in the path of the light with a first light filter, where the first light filter directs filtered light to a first camera recording a first indicator of cardiac physiology. There is a second filter cube in the path of the light with a second light filter, where the second light filter directs filtered light to a second camera recording a second indicator of cardiac physiology. There is also a third light filter in the path of the light, where the third light filter directs filtered light to a third camera recording a third indicator of cardiac physiology. Using those recordings, the system can map cardiac physiology simultaneously.

Herein, we report a new approach to simultaneously image Vₘ, Ca²⁺ and NADH (metabolic marker). Optical mapping is a methodology that optically records cardiac physiology with high spatial and temporal resolution, either as autofluorescence of endogenous biological substances or as fluorescence of specifically designed dyes. Optical mapping of the heart was first applied to record Vₘ and then NADH autofluorescence. Since then, optical mapping has also been applied to record Ca²⁺. Dual parameter optical mapping of Vₘ and NADH as well as Vₘ and Ca²⁺ have also been applied in assessing cardiac physiology. However, as described above, the three interdependent facets of cardiac function will all need to be measured simultaneously to develop a complete picture of cardiac physiological modulation by drugs or disease. In this study, we report for the first time, a spatially and temporally co-registered triple-parametric optical mapping system that incorporates three cameras to simultaneously capture NADH, Vm, and Ca²⁺ signals from the same field of view.

In certain embodiments, the technology comprises a second lens positioned between the first light filter and the first camera.

In other embodiments, the technology comprises a third lens positioned between the second light filter and the second camera.

In embodiments, the technology comprises using the parametric mapping to analyze metabolism-excitation-contraction coupling in cardiac tissue.

In yet other embodiments, the first, second, and third indicators of cardiac physiology are NADH, calcium, and voltage.

In embodiments, the first excitation light source has a wavelength of approximately 520 nm and the second excitation source has a wavelength of approximately 365 nm.

In other embodiments, the system further generates one or more activation maps and one or more intensity maps from the first, second, and third indicators of cardiac physiology.

In certain embodiments, the system simultaneously measures up to ten physiological parameters during parametric mapping.

In yet other embodiments, up to ten parameters are related to repolarization and calcium reuptake.

In other embodiments, the parametric mapping is used to indicate drugs effects or sequence of modulation of cardiac physiology in a disease of the cardiac tissue.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1A is a diagram showing a schematic of a triple parametric optical mapping system illustrating the optics used in the system and the light path for the three signals—Vₘ, Ca²⁺ and NADH;

FIG. 1B is a 3D rendering of the triple parametric optical mapping system illustrating the 3D printed hardware that houses the optics used in an exemplary embodiment of the system;

FIG. 1C is a spectra of the three parameter signals: NADH autofluorescence, Rhod2-AM (Ca) and RH237 (Vₘ) fluorescence, illustrating the separation of the three signals as implemented in this system. Solid vertical lines: LED light source wavelength, dotted vertical lines: dichroic mirror, boxes: filters. Illustration of the different parameters measured using the three optical signals;

FIG. 1D is a diagram showing how NADH intensity was determined and each intensity value from a given heart was normalized to the NADH intensity from the first recording from that heart;

FIG. 1E is a diagram showing the definition of parameters measured from the depolarization phase of the Vₘ signal or the calcium release phase of the Ca²⁺ signal;

FIG. 1F is a diagram showing the definition of the parameters measured from the repolarization phase of the Vₘ signal or the calcium reuptake phase of the Ca²⁺ signal;

FIG. 2A shows electromechanical uncoupling in triple parametric optical mapping.

Representative traces of Vₘ and Ca²⁺ (solid and dotted traces, respectively) recorded without (Control, black) and with the electromechanical uncoupler Blebbistatin (15 μM, orange);

FIG. 2B shows representative Vₘ activation maps (left), Ca²⁺ activation maps (middle) and NADH intensity maps (right) recorded during Control and Blebbistatin treatment. All maps were recorded from the same heart, the top three maps were from simultaneous recording during Control and the bottom three from simultaneous recording during Blebbistatin treatment. Slightly different silhouettes, particularly around the boundaries is due to different background noise removal levels using a thresholding algorithm. These boundary pixels were not used in analysis;

FIG. 2C are graphs showing the summary restitution properties of the ten parameters measured simultaneously by triple parametric optical mapping, in accordance with an exemplary embodiment of the present technology. Least squares regression analysis was performed to detect significant differences in parameters during Control versus Blebbistatin treatment and all significant p values are reported. Benjamini-Hochberg correction was applied to account for multiple comparisons;

FIG. 2D is a ten parameter panel showing the effects of Blebbistatin on cardiac physiology at 150 ms basic cycle length, reported as percent change from Control. Data presented as mean±s.e.m., n=5 hearts, Two-tailed, paired t-tests were performed to detect significant change induced by Blebbistatin compared to Control and all significant p values are reported. Benjamini-Hochberg correction was applied to account for multiple comparisons;

FIG. 3A shows ten-parameter panels demonstrating effects of 4-AP (7 mM, left, green) and Verapamil (1 μM, right, magenta). Two-tailed, paired t-tests were performed to detect significant change induced by drugs compared to Blebbistatin and all significant p values are reported. Benjamini-Hochberg correction was applied to account for multiple comparisons;

FIG. 3B shows graphs that display summary restitution properties of the ten parameters measured simultaneously by triple parametric optical mapping. Data presented as mean±s.e.m., n=5 hearts. Least squares regression analysis was performed to detect significant differences in parameters during Blebbistatin versus drugs (4-AP, Verapamil) treatment and all significant p values are reported. Benjamini-Hochberg correction was applied to account for multiple comparisons;

FIG. 4A shows triple parametric optical mapping in cardiac disease assessment. Representative Vₘ activation maps (top), Ca²⁺ activation maps (middle) and NADH intensity maps (bottom) recorded from the same mouse heart during baseline, ischemia and reperfusion conditions. All maps in a given column were obtained by simultaneous imaging;

FIG. 4B shows graphs that display summary restitution properties of seven parameters measured simultaneously by triple parametric optical mapping. Three parameters—APD₈₀, CaTD₈₀ and Ca²⁺ decay were not measurable due to motion artifacts in the repolarization phase due to absence of electromechanical uncoupling. The values reported are normalized to baseline (t=0) for each heart in order to determine modulation of each parameter in each heart with respect to its own baseline condition;

FIG. 4C shows ten parameter panels (only 7 parameters reported) demonstrating changes in cardiac physiology during ischemia and restoration during reperfusion. Data presented as mean±s.e.m., n=5 hearts. Two-tailed, paired t-tests were performed to detect significant change induced by ischemia and reperfusion compared to baseline and all significant p values are reported. Benjamini-Hochberg correction was applied to account for multiple comparisons.

FIG. 5A is a map showing cardiac electrical, calcium handling and metabolic response to exercise. Simultaneously recorded NADH intensity maps and voltage and calcium activation maps are generated from triple-parametric optical mapping. All three maps in a given column are simultaneous recordings of the same field of view;

FIG. 5B is a graph showing representative voltage and calcium traces from optical mapping of hearts in the four experimental groups. Sedentary: solid line, exercised: dashed lines; and

FIG. 5C is a chart showing ten parameter panel illustrating changes in transmembrane potential, calcium handling and metabolism related parameters. Average values of percentage change in exercised value versus sedentary controls are reported for each parameter.

### DETAILED DESCRIPTION OF THE PREFERRED EMBODIMENTS

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes, it being understood that the invention may be embodied in other forms not specifically shown in the drawings.

Preclinical safety and efficacy testing are a crucial component of the drug development process. This step is important in determining dosing and toxicity which could include identifying off-target effects of drugs before clinical trials and approval for use in patients. Cardiotoxicity is the primary cause (19%) of drug withdrawal from the market in the United States and the second leading cause worldwide, underscoring the need for efficient and thorough cardiac methodologies of drug screening. Current preclinical drug testing primarily focuses on the effects of drugs on the electrical activity (QT interval) and contractility of the heart. While this is an essential first step, it does not give a complete picture of cardiac physiology modulation by drugs as it does not consider calcium handling or metabolic states of the cardiac tissue. Unexpected off target effects of the drugs being tested could result in serious complications or fatality. Here, we present a novel approach to measure ten different important aspects of cardiac MECC using triple parametric optical mapping. We investigated the effects of three different compounds (blebbistatin, 4-aminopyridine and verapamil) using triple parametric optical mapping and present the data in a ten-parameter panel (TPP). TPP graphs include information on action potential upstroke, duration and conduction, intracellular calcium release and reuptake as well as the metabolic state of the heart.

Triple parametric optical mapping and TPP graphs may also benefit the study of complex cardiac diseases such as ischemia and reperfusion. Acute ischemic bouts are known to have multiple and severe effects on cardiac physiology. Ischemia has been previously demonstrated to alter the electrical activity, calcium handling as well as the metabolic functions of the heart. However, the sequence and the interrelationship between these three aspects of cardiac MECC have not been studied simultaneously before, due to lack of appropriate methodology. Here, we also determined the simultaneous modulation of multiple aspects of cardiac physiology by ischemia and their restoration during reperfusion. Thus, applying triple parametric optical mapping to study MECC during disease progression could provide valuable new targets for therapy.

Hardware Design

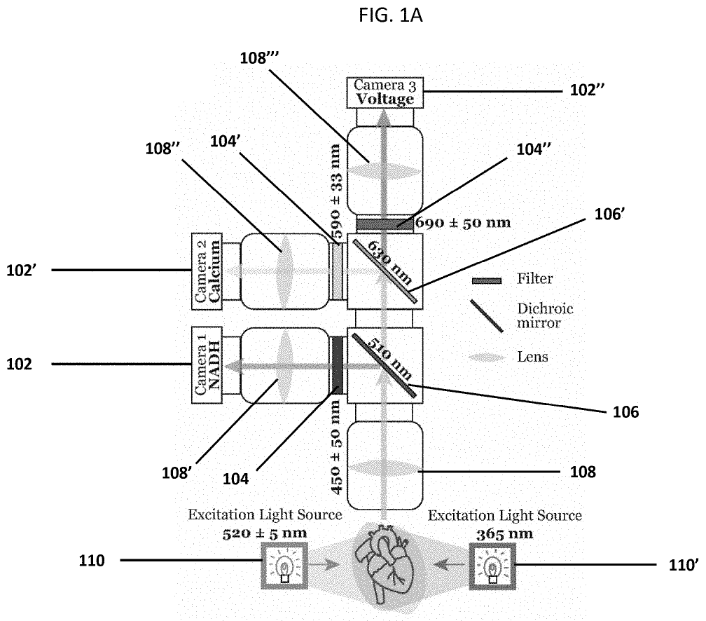

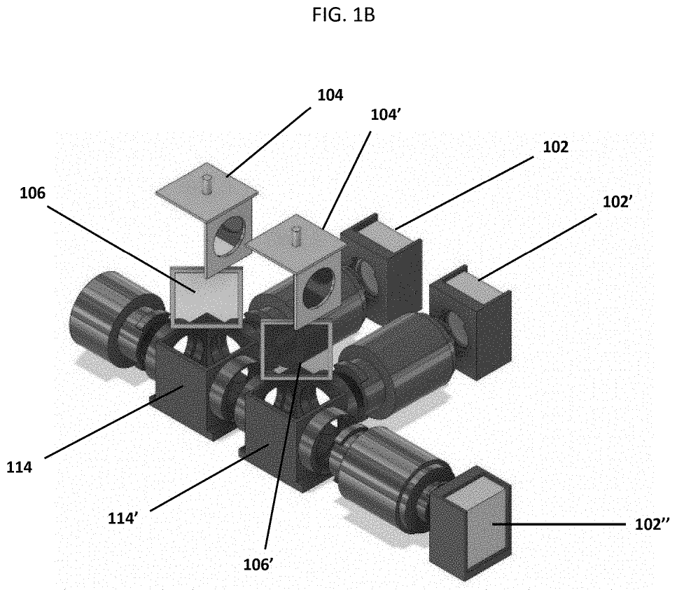

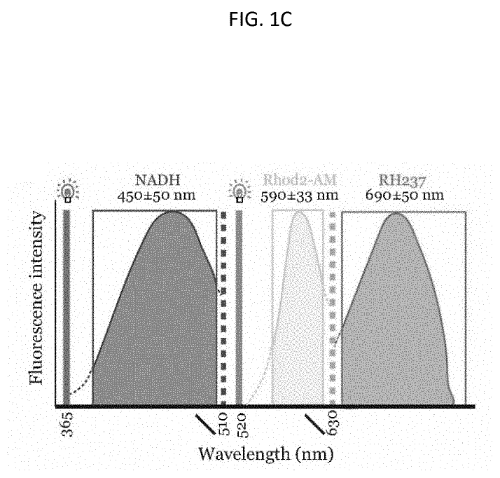

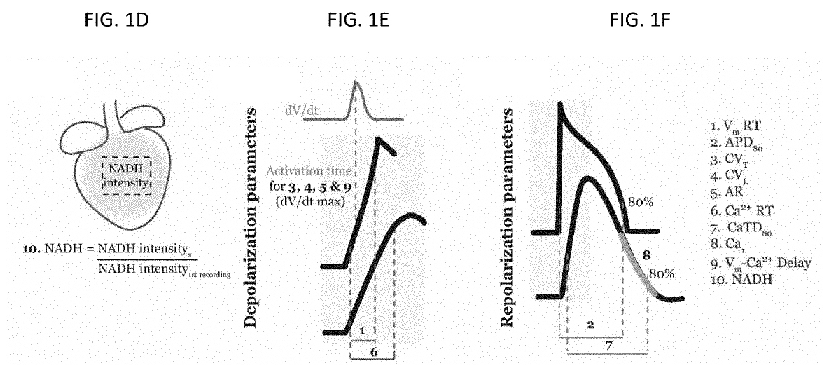

FIGS. 1A and 1B show a schematic and 3D rendering of an exemplary embodiment of the triple parameter optical mapping system.

Triple Parametric Optical Mapping System Set Up and Alignment

The triple parametric optical mapping system uses three CMOS cameras 102, 102′, 102″ (MiCAM05, SciMedia) and the MiCAM05 digital interface for 3-4 camera (SciMedia). BV workbench 2.6.1 (SciMedia) was the software used for camera alignment and acquisition. This system triggers all attached cameras 102, 102′, 102″ simultaneously. The cameras 102, 102′, 102″ have 100×100 pixel resolution with a camera sensor of 10×10 mm dimension. In certain embodiments, a sampling time of 1 ms (1 kHz frequency) was used to acquire the data file of 2 s duration.

All cameras 102, 102′, 102″ are focused on the same field of view through a tandem lens 108, 108′, 108″, 108′″ configuration as illustrated in FIGS. 1A and 1B. All filter cubes 114, 114′, mechanical and stage components of the system were custom 3D printed and system assembly instructions were previously published for dual parameter optical mapping, Cathey B, Obaid S, Zolotarev A M, Pryamonosov R A, Syunyaev R A, George S A, Efimov I R. Open-Source Multiparametric Optocardiography. Sci Rep, 9: 721. 2019. The camera cages were specifically designed for SciMedia cameras but the other system components can be used with optical mapping systems from other sources. Two modifications were made to these parts to accommodate an extra camera in this current system (FIG. 1B). Open-source files of these components are also available at Github (https://github.com/optocardiography).

Two light emitting diode (LED) excitation light sources 110, 110′ at 365 nm (Mightex Systems, LCS-0365-04-22) and 520±5 nm (Prizmatix, UHP-Mic-LED-520) with collimators were used in episcopic illumination mode to excite the dyes or induce autofluorescence in the cardiac tissue. The emitted light is collected by an infinity-corrected, planapo 1× lens 108 (objective lens, SciMedia) with a working distance of 61.5 mm. This lens 108 then focuses the collected light at infinity. This infinity correction allows multiple cameras 102, 102′, 102″ to be introduced in the light path. Light at different wavelengths were separated using dichroic mirrors 106, 106′ and filters 104, 104′, 104″ as illustrated in FIG. 1C and passed through a second planapo 1× lens 108′, 108′, 108′″ (projection lens, SciMedia) before being recorded using the CMOS cameras 102, 102′, 102″. The use of two lenses 108/108′, 108/108″, 108/108′″ of the same focal length in tandem lens configuration results in a magnification of 1× (pixel size=0.1 mm). First, light below 510 nm is split from the straight light path by a dichroic mirror 106 which is then filtered by a 450±50 nm filter 104 and directed to the first CMOS camera 102 to record NADH autofluorescence. Next, Rhod2-AM (intracellular calcium) signal is split from the straight light path using a 630 nm dichroic mirror 106′, filtered using a 590±33 nm filter 104′ and recorded using the second CMOS camera 102′. Finally, the RH237 (transmembrane potential) signals at wavelengths above 630 nm are filtered through a 690±50 nm filter 104″ and recorded using the third CMOS camera 102″. With this optical system, the amount of emission spectral overlap between NADH and Rhod2-AM as well as between Rhod2-AM and RH237 can be reduced and the interference from a different channel is minimized.

At the start of each experiment, all three cameras 102, 102′, 102″ were focused and aligned. Each camera 102, 102′, 102″ was attached to its own projection lens 108′, 108″, 108′″ and focused at infinity. The projection lens/camera unit was then attached to the filter cubes 114, 114′. A focusing target was positioned in front of the objective lens and the position of the target was adjusted until all three cameras were in focus. Next, the three cameras 102, 102′, 102″ were spatially aligned using the Camera Calibration function in the BVWorkbench software (Brainvision). This feature overlays the image captured by the different cameras and uses an edge detection algorithm to allow the user to manually adjust the angle of the dichroic mirrors 106, 106′ until all fields of view are spatially aligned. Once, all cameras 102, 102′, 102″ were focused and aligned the system is ready for use.

Langendorff perfusion: Adult male and female mice on C57BL/6 background were used in this study. Mice were anesthetized by isoflurane inhalation and cervical dislocation was performed. Hearts were quickly excised following thoracotomy and the aorta was cannulated. The heart was attached to a Langendorff perfusion system, hung in a vertical position in a temperature-controlled bath and perfused with a modified Tyrode's solution containing (in mM) 130 NaCl, 24 NaHCO₃, 1.2 NaH2PO4, 1 MgCl2, 5.6 Glucose, 4 KCl, 1.8 CaCl2, pH 7.40 and bubbled with carbogen (95% 02 and 5% CO2) at 37° C. Perfusion pressure was maintained at ˜80 mmHg by adjusting the flow rate between 1 and 1.5 ml/min. A platinum bipolar electrode was placed at the center of the anterior surface residing the middle of the field of view. Gentle pressure was applied to the back of the heart as it was pushed up to the front optical glass of the bath using a paddle, allowing the pacing electrode to be held in place. Electrical stimuli were applied to determine the threshold of pacing. Hearts were paced at 1.5× pacing threshold amplitude and 2 ms stimulus duration, only during optical recording. Hearts were paced at varying rates as listed in the figures to determine the restitution properties of the heart under each condition.

Optical Mapping: The heart was subjected to a 10 min equilibration period followed by staining with the voltage- and calcium-sensitive dyes. A 1 ml mixture of RH237 (30 ml of 1.25 mg/ml dye stock solution+970 ml Tyrode's solution, Biotium 61018) was prepared and immediately injected into the dye port above the cannula over a 3-5 min period. This was followed by a 5 min washout period. Similarly, a 1 ml mixture of Rhod2-AM (30 ul of 1 mg/ml dye stock solution+30 ml Pluronic F-127+940 ml Tyrode's solution, Thermo Fisher Scientific R1244 and Biotium 59005, respectively) was prepared and immediately injected into the dye port over a 3-5 min period. This was followed by 5 min dye washout period.

The heart was then illuminated by two LED excitation light sources at 365 nm and 520±5 nm wavelengths. While the former induces autofluorescence of NADH in the tissue, the latter excites both RH237 and Rhod2-AM dyes. Control optical recordings of NADH, Vₘ, and Ca²⁺ were simultaneously acquired at 1 kHz sampling rate. NADH, Vₘ, and Ca²⁺ are indicative of contraction, excitation and metabolism. The simultaneous recording of these three parameters by the present technology allows the determination of MECC.

Electromechanical uncoupling and drug treatment: After Control recordings, hearts were treated with 15 mM blebbistatin (Cayman Chemicals 13186) for 20 mins and optical recordings were acquired as above. Next, hearts were treated with 4-AP (7 mM, Millipore Sigma 275875) and verapamil (1 mM, Sigma Aldrich V4629) one at a time, in the presence of blebbistatin. Optical recordings were once again acquired for each of these conditions as described above.

Ischemia: In a separate set of hearts, after the initial equilibration period and dye staining/washout, hearts were subjected to 5 mins of no flow ischemia followed by 5 mins of reperfusion. Hearts were continually paced at 300 ms BCL throughout this period. Optical recordings were acquired prior to (baseline), during (ischemia) and after (reperfusion) this period of no perfusion at 1 min intervals. Ischemia period was limited to 5 mins because beyond this point, the optical signals were of very poor quality which would not allow suitable analysis. Signal quality deteriorates during periods of ischemia due to poor or no perfusion of the tissue and quickly recovers during reperfusion. Poor quality signals are defined as those with low signal-to-noise ratios that would not allow for the accurate measurements of the parameters.

Data Analysis: Optical data of NADH, transmembrane potential and calcium were analyzed using a custom Matlab software, Rhythm 3.0, which is available in an open-source format on Github. Rhythm 3.0 which is an upgraded version of Rhythm 1.2, incorporates NADH visualization and analysis features as well as the ability to calculate delay in transmembrane potential to intracellular calcium activation (Vₘ-Ca²⁺ Delay). Rhythm software was written to analyze data formats generated by SciMedia systems but can be modified to analyze other data formats.

Three optical signal recordings of NADH, Vₘ, and Ca²⁺ are simultaneously and dynamically collected in real time using the device of the present technology as follows. First, the device passes light of light sources emitting different wavelengths of light to a cardiac tissue, preferably at a wavelength of 520±5 nm 110 and 365 nm 110′. The light passes through a lens 108 and a filter cube 114 that contains a dichroic mirror 106, which preferably reflects light with a wavelength of 510 nm. The mirror 106 reflects the light to a filter 104, preferably filtering at a wavelength of 450±50 nm through a lens 108′ to a first camera 102 that outputs an optical recording of NADH. Light also passes to a second filter cube 114′ that contains a second mirror 106′, which preferably reflects light with a wavelength of 630 nm. The mirror 106′ reflects the light to a filter 104, preferably filtering at a wavelength of 590±33 nm through a lens 108″ to a second camera 102′ that outputs an optical recording of Ca²⁺. In the second filter cube 114′, the light also passes through a third filter 104″, preferably filtering at a wavelength of 690±50 nm, and through a lens 108′″ to a third camera 102″ that outputs an optical recording of Vₘ. The three recorded values are mapped simultaneously. The first, second and third cameras 102, 102′, 102″ are each a sensor. The cameras 102, 102′, 102″ do not transform the signals, and any suitable sensor can be used other than a camera. In some embodiments, the cameras 102, 102′, 102″ can each include a memory and record the optical signal as a video file to the memory.

Ten different parameters were measured from the 3 optical signals that were simultaneously recorded under each condition using the Rhythm 3.0 software to determine MECC. The ten parameters include, from transmembrane potential: (1) rise time (Vₘ RT), (2) action potential duration (APD₈₀), (3) transverse and (4) longitudinal conduction velocity (CV_{T} and CV_{L}) and (5) anisotropic ratio (AR), from intracellular calcium: (6) rise time (Ca²⁺ RT), (7) calcium transient duration (CaTD₈₀), (8) calcium decay time constant (t), (9) Vₘ-Ca²⁺ delay and (10) NADH fluorescence intensity. The rate-dependence of each of these parameters is indicated in the parameter vs BCL graphs while the summary of effects of each condition is illustrated in the TPP graphs. The values reported in the TPP graphs correspond to BCL=150 ms which is considered “normal” heart rate (400 bpm) for an ex vivo mouse heart preparations.

Upstroke rise time (RT) from Vₘ and Ca²⁺ signals were measured as the time period from 20 to 90% of the upstroke of the action potential and calcium transient, respectively. APD₈₀ and CaTD₈₀ were measured as the time interval between activation time (time of maximum first derivative of the upstroke) and 80% of repolarization and calcium transient decay, respectively. CV_{L} and CV_{T} in the parallel and perpendicular direction to fiber orientation, respectively, were calculated using differences in activation times and known interpixel distances. Anisotropic ratio was calculated as the ratio of CV_{L} to CV_{T}. Calcium decay time constant (t) was determined by fitting an exponential to the last 50% (50-100%) of the calcium transient decay phase. Vₘ-Ca²⁺ delay was defined as the time interval between the activation time of the Vₘ signal minus the activation time of the Ca²⁺ signal. Lastly, NADH intensity was measured as the average of the absolute autofluorescence intensity value in the optical recording. NADH intensity in each heart was normalized to the first measured NADH intensity value from that particular heart in order to avoid interexperimental variability.

In certain embodiments, a processing device can be provided, together with a memory and a light sensor. The processing device and/or memory can be located locally at the sensor, so that each sensor is associated with a respective processing device. Or a single processing device can be provided and centrally located and in wired or wireless communication with each sensor, and a central memory can be in communication with the processing device; and/or each sensor can be associated with its own memory. The sensors each detect the respective one of the three (3) optical signals and save the signal to memory. The processing device can determine the ten parameters in real time as it is received by the sensor, or it can retrieve the optical signals from memory and determine the ten (10) parameters. The processing device can then use the parameters to generate a report, generate an alarm, or a control signal that controls a machine or device such as a medical device to dynamically impart or adjust therapy to a patient such as for example pacing or stimulation.

The processing device can be a processor, controller, computer, server, tablet, smartphone, or the like. The processing device can be used in combination with other suitable components, such as a display device (monitor, LED screen, digital screen, etc.), memory or storage device, input device (touchscreen, keyboard, pointing device such as a mouse), wireless module (for RF, Bluetooth, infrared, WiFi, etc.). The information may be stored on a computer medium such as a computer hard drive, or on any other appropriate data storage device, which can be located at or in communication with the processing device. The entire process is conducted automatically by the processing device, such as by software stored at the memory (e.g., Matlab), and without any manual interaction. Accordingly, unless indicated otherwise the process can occur dynamically and substantially in real-time without any delays or manual action. The processing device can be used to implement the operations described above and/or below.

Statistics & Reproducibility: All data are reported as mean±standard error of the mean (SEM). A sample size of 5 hearts was used for all groups, drug treatment and ischemia protocols. An alpha level of 0.05 was used in all tests.

Regression analysis was performed on the restitution data using Graphpad Prism Version 9.3.1 software. Non-linear regression analysis was performed using the least squares regression fitting method. For parameters that exhibited restitution property (CV_{T}, CV_{L}, AR, APD₈₀, CaTD₈₀, and Ca t), an exponential plateau model (Y=Y_{M}−(Y_{M}−Y₀) e^{-kX}) was used. For Vm-Ca delay parameter, third order polynomial model (Y=B₀+B₁X+B₂X²+B₃X³) was used. For all others a simple linear regression model (Y=α+bX) was used. All other parameters were left at default settings in this software. Good model fit was determined by r-squared value>0.5 (except Vm-Ca delay during blebbistatin treatment) and by confirming the random nature of the residual plots.

The parameters of the best fit were compared between groups (Control vs Blebbistatin, Blebbistatin vs 4-AP, Blebbistatin vs Verapamil) using the extra sum-of-squares F test. Two-tailed paired t-tests were performed for all other data. Since multiple statistical tests were performed on this data set (10 parameters, 5 treatments), Benjamini-Hochberg correction was applied with a false discovery rate of 20%. Shapiro-Wilk test was applied to test for data normality of the raw data values as well as the best-fit parameters of the regression analysis and >95% data sets passed the normality test. However, the small sample size of this data set may be a limitation.

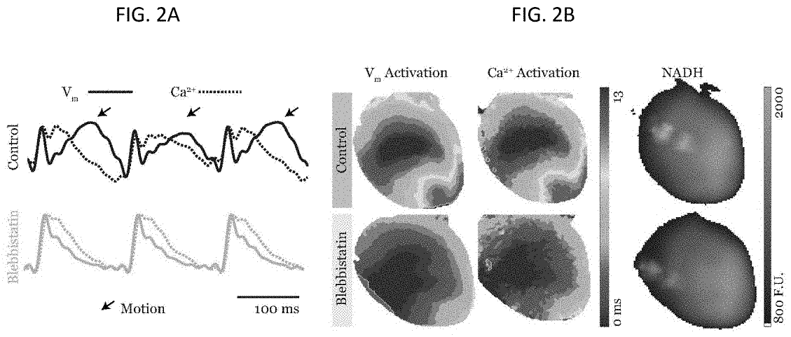

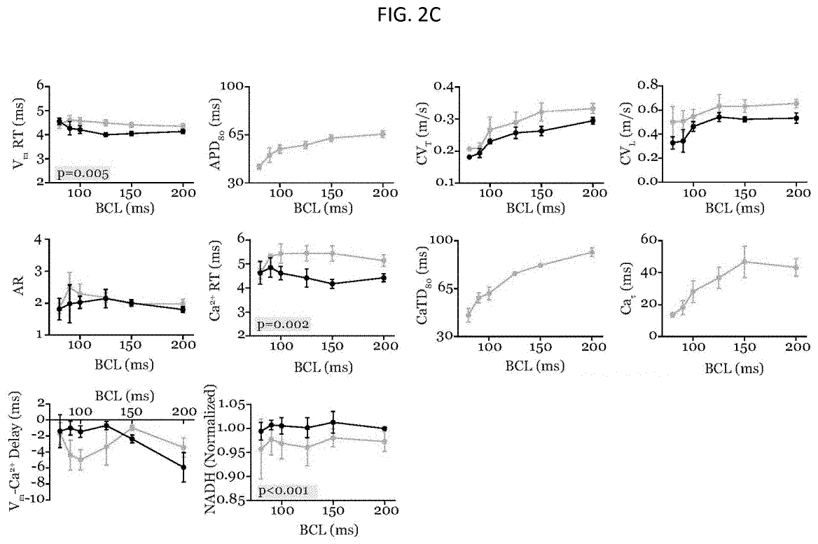

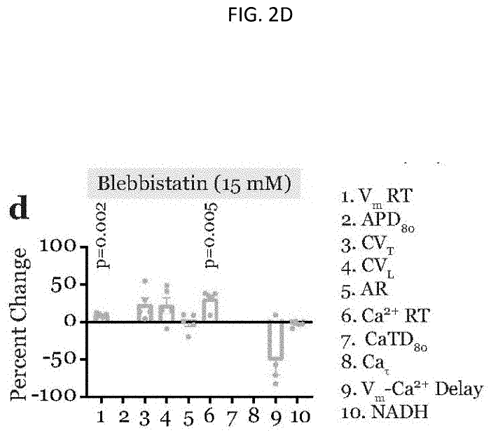

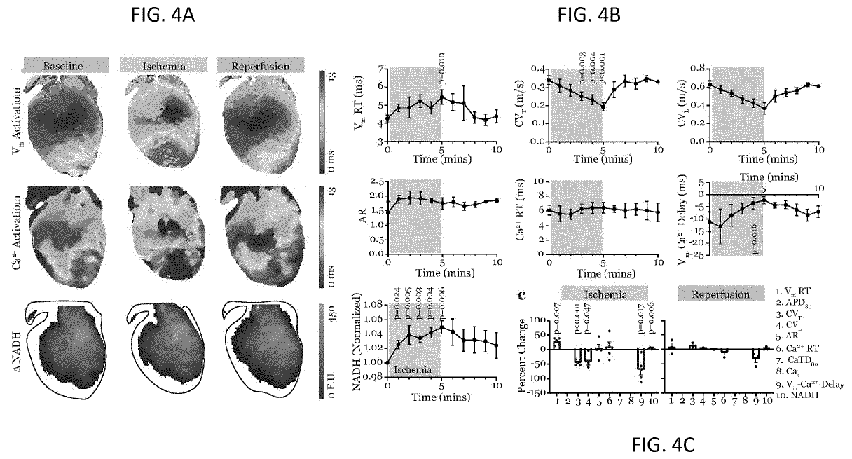

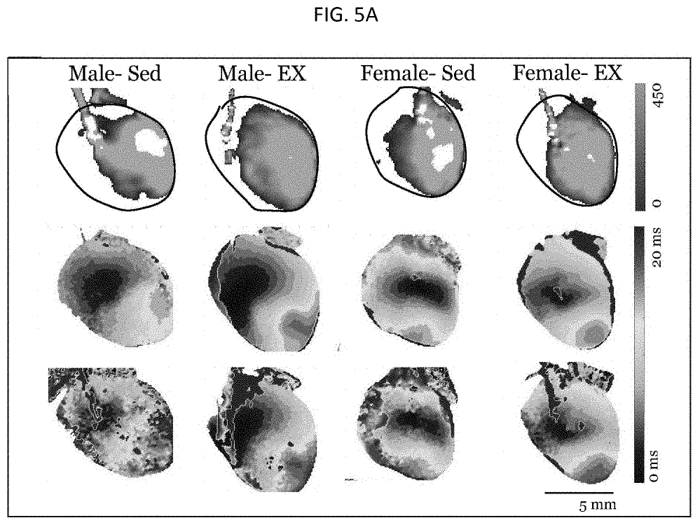

For the summary data in FIGS. 2D, 3A and 4, paired two-tailed t-tests were performed. In the case of FIGS. 2D, 3A and 4C, percent change in each parameter by a given drug (Blebbistatin, 4-AP and Verapamil) versus Control was compared. In FIG. 4B, statistical tests compared change in each parameter at a given time point versus Baseline (t=0). Benjamini-Hochberg correction was applied with a false discovery rate of 20% to account for multiple comparisons.

Results

The triple parametric optical mapping system was 3D printed and set up as illustrated in FIGS. 1A and 1B, and the separation of signals of different wavelengths is illustrated in FIG. 1C. All design files for 3D printed hardware (in STL format) and data analysis software (Matlab) are available under an open-source license at Github (https://github.com/optocardiography, DOI: 10.5281/zenodo.5784023). The following 10 parameters were measured from the optical recordings and an illustration of each parameter definition is included in FIGS. 1D-1F. From the NADH recordings, the absolute intensity of the NADH signals was measured which corresponds to NADH concentration in the tissue. Since this parameter can vary between hearts depending on experimental conditions, all measurements from a given heart were normalized to the first recording from that same heart (Control at 200 ms pacing rate for drug testing protocol and Baseline at 200 ms pacing rate for ischemia protocol). This allows the determination of changes in NADH induced by drug treatment or disease without confounding interexperimental variables.

From the depolarization/calcium release phase of the Vₘ and Ca signals, Vₘ and Ca Rise Times (Vₘ RT and Ca RT), longitudinal and transverse conduction velocity (CV_{L} and CV_{T}), anisotropic ratio (AR), and activation delay between Vₘ and Ca traces (Vₘ-Ca delay) were calculated. Rise time was defined as the time taken for depolarization, from 20 to 90%. In the case of Vₘ RT, this parameter indicates function of depolarizing currents while in the case of Ca RT, this parameter indicates time taken for calcium entry into the cell and calcium-induced calcium release from the sarcoplasmic reticulum. Conduction velocity is the speed with which the activation wavefront travels in a given direction, longitudinal (parallel to fiber orientation) and transverse (perpendicular to fiber orientation). AR is the ratio of CV_{L} to CV_{T} and indicates ellipticity of the propagating wavefront.

Higher AR is associated with increased arrhythmogenicity. Vₘ-Ca delay is calculated to determine the excitation-contraction coupling. Prolonged delay suggests uncoupling between electrical excitation and mechanical contraction. From the repolarization/calcium reuptake phase of the Vₘ and Ca signals, action potential duration at 80% repolarization (APD₈₀), calcium transient duration at 80% reuptake (CaTD₈₀), and calcium decay constant (Ca t) were calculated. It is important to note that these 3 parameters (APD₈₀, CaTD₈₀ and Ca t) were only measurable in hearts after Blebbistatin perfusion. Without Blebbistatin, motion artifacts were present in the signals which distort the Vₘ and Ca signals in the repolarization/reuptake phase (FIG. 2A, top). APD₈₀ was defined as the time interval between activation and repolarization or in other words the duration for one cardiac cycle. Similarly, CaTD₈₀ was defined as the time interval between calcium release and reuptake of calcium back into the sarcoplasmic reticulum. Both shortening and prolongation of these parameters have been reported to be arrhythmogenic. Lastly, Ca t is the decay constant measured by fitting an exponential to the reuptake phase of the calcium transients. This parameter is indicative of how quickly calcium in the cytoplasm is removed after each contraction.

Blebbistatin Modulates Cardiac Physiology

Motion of the heart during image acquisition introduces artifacts in the recorded optical signals which hinder the analysis of repolarization-related parameters. To prevent these motion artifacts, electromechanical uncouplers such as blebbistatin are routinely used in optical mapping of Vₘ and Ca²⁺. Thus, the first step in this study was to analyze the effects of blebbistatin on the ten parameters of cardiac physiology.

Representative Vₘ and Ca²⁺ traces (FIG. 2A, top) illustrate motion artifacts that are introduced in the repolarization phases in Control (no treatment) hearts. Activation/intensity maps generated from these traces are shown in FIG. 2B (top). Optical recordings during Control treatment allowed for the measurement of 7 parameters —Vₘ RT, CV_{T}, CV_{L}, AR, Ca²⁺ RT, Vₘ—Ca²⁺ delay, and NADH. Restitution, which is the property of electrophysiological parameters to vary with diastolic interval (typically decrease with decreasing diastolic interval), was observed in CV_{T} and CV_{L}.

Treatment with blebbistatin (15 mM) abolished contractions and removed motion artifacts as shown in FIG. 2A (bottom). Preventing motion-induced distortion of signals in the later phases of the action potential and calcium transient allowed the measurement of repolarization/calcium reuptake parameters such as APD₈₀, CaTD₈₀ and Ca t. Restitution property was observed in APD₈₀, CV_{T} and CV_{L}. Additionally, CaTD₈₀ and Cat was also rate dependent. Specifically, both CaTD₈₀ and Cat decreased with increasing pacing rate (FIG. 2C).

Blebbistatin also induced significant differences in cardiac physiology compared to Control. Blebbistatin increased both Vₘ and Ca²⁺ RTs (p=0.005 and 0.002, respectively). On the other hand, NADH intensity was reduced after blebbistatin treatment (p<0.001, FIG. 2C).

In the TPP graph in FIG. 2D, percent change in each of the ten parameters induced by blebbistatin with respect to Control, at 150 ms BCL (basic cycle length), is summarized which once again illustrates that blebbistatin significantly increases Vₘ and Ca²⁺ RTs (p=0.002 and 0.005, respectively).

On- and Off-Target Effects of Drugs on Cardiac Physiology

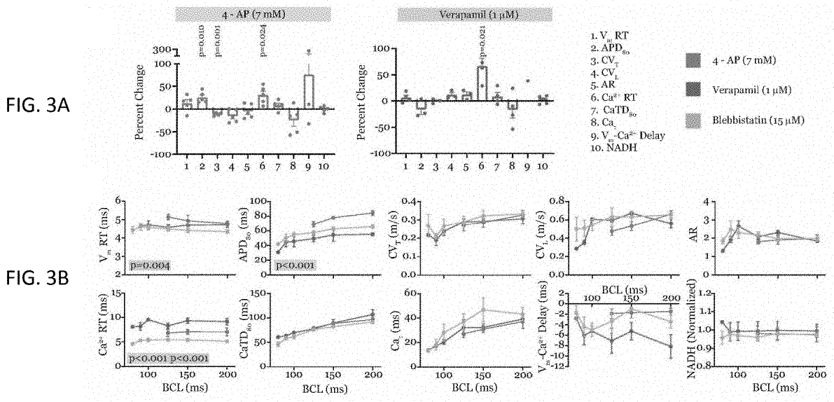

The application of triple parametric optical mapping in drug testing was then evaluated with two well studied drugs currently used in treating patients—4-AP and verapamil. The effects of these drugs on cardiac physiology were tested in the presence of blebbistatin to be able to determine repolarization-/calcium reuptake-related parameters. Therefore, each physiological parameter during 4-AP and verapamil treatment were compared to blebbistatin treatment to determine significant drug-related effects. The effects of 4-AP and verapamil on cardiac physiology are summarized in FIGS. 3A-3B and in Table 1. The TPP graphs in FIG. 3A, demonstrate the differences between 4-AP and verapamil. While 4-AP had multiple on-target and off-target effects on cardiac physiology, verapamil only had a specific on-target effect.

TABLE 1
Modulation of cardiac physiology by Blebbistatin (15
μM), 4-AP (7 mM) and Verapamil (1 μM).

| BCL | Control | Blebbistatin | 4-AP | Verapamil |
|---|---|---|---|---|
| 200 | 4.13 ± 0.17 | 4.34 ± 0.20 | 4.79 ± 0.23 | 4.72 ± 0.28 |
| 150 | 4.04 ± 0.17 | 4.40 ± 0.21 | 4.94 ± 0.63 | 4.70 ± 0.46 |
| 125 | 3.99 ± 0.11 | 4.48 ± 0.20 | 5.15 ± 0.29 | 4.57 ± 0.25 |
| 100 | 4.20 ± 0.28 | 4.56 ± 0.28 | | 4.70 ± 0.39 |
| 90 | 4.26 ± 0.39 | 4.60 ± 0.32 | | 4.65 ± 0.10 |
| 80 | 4.55 ± 0.12 | 4.43 ± 0.24 | | |
| 200 | | 65.62 ± 4.93 | 84.56 ± 6.68 | 55.28 ± 2.94 |
| 150 | | 62.67 ± 4.70 | 78.13 ± 3.52 | 54.39 ± 11.88 |
| 125 | | 56.27 ± 4.56 | 69.09 ± 7.52 | 49.46 ± 6.20 |
| 100 | | 54.77 ± 5.95 | | 45.75 ± 10.51 |
| 90 | | 50.38 ± 9.10 | | 44.72 ± 9.21 |
| 80 | | 42.06 ± 2.68 | | 31.06 ± 0.00 |
| 200 | 0.29 ± 0.02 | 0.33 ± 0.03 | 0.32 ± 0.05 | 0.30 ± 0.05 |
| 150 | 0.26 ± 0.02 | 0.32 ± 0.05 | 0.28 ± 0.05 | 0.29 ± 0.01 |
| 125 | 0.25 ± 0.02 | 0.28 ± 0.05 | 0.27 ± 0.05 | 0.28 ± 0.03 |
| 100 | 0.23 ± 0.01 | 0.26 ± 0.07 | | 0.23 ± 0.03 |
| 90 | 0.19 ± 0.01 | 0.21 ± 0.02 | | 0.18 ± 0.04 |
| 80 | 0.18 ± 0.00 | 0.26 ± 0.08 | | 0.21 ± 0.00 |
| 200 | 0.53 ± 0.08 | 0.65 ± 0.06 | 0.66 ± 0.06 | 0.56 ± 0.09 |
| 150 | 0.52 ± 0.04 | 0.63 ± 0.10 | 0.53 ± 0.09 | 0.67 ± 0.02 |
| 125 | 0.54 ± 0.05 | 0.63 ± 0.16 | 0.47 ± 0.07 | 0.59 ± 0.03 |
| 100 | 0.46 ± 0.06 | 0.54 ± 0.12 | | 0.60 ± 0.02 |
| 90 | 0.34 ± 0.09 | 0.50 ± 0.15 | | 0.35 ± 0.03 |
| 80 | 0.32 ± 0.05 | 0.50 ± 0.18 | | 0.28 ± 0.00 |
| 200 | 1.80 ± 0.18 | 1.98 ± 0.27 | 1.97 ± 0.37 | 1.83 ± 0.12 |
| 150 | 2.00 ± 0.19 | 1.97 ± 0.19 | 1.90 ± 0.45 | 2.30 ± 0.14 |
| 125 | 2.14 ± 0.39 | 2.17 ± 0.26 | 1.80 ± 0.19 | 2.07 ± 0.21 |
| 100 | 2.02 ± 0.32 | 2.29 ± 0.61 | | 2.64 ± 0.51 |
| 90 | 1.98 ± 0.59 | 2.47 ± 0.82 | | 1.92 ± 0.23 |
| 80 | 1.81 ± 0.33 | 1.84 ± 0.22 | | 1.31 ± 0.00 |
| 200 | 4.41 ± 0.32 | 5.12 ± 0.47 | 7.04 ± 1.57 | 9.14 ± 1.31 |
| 150 | 4.16 ± 0.37 | 5.43 ± 0.64 | 7.05 ± 1.03 | 9.31 ± 0.93 |
| 125 | 4.40 ± 0.53 | 5.44 ± 0.55 | 6.82 ± 1.39 | 8.28 ± 0.96 |
| 100 | 4.60 ± 0.47 | 5.42 ± 0.81 | | 9.54 ± 0.43 |
| 90 | 4.84 ± 0.57 | 5.30 ± 0.15 | | 8.16 ± 1.36 |
| 80 | 4.62 ± 0.46 | 4.58 ± 0.30 | | 8.09 ± 0.00 |
| 200 | | 91.55 ± 6.57 | 96.41 ± 8.18 | 106.68 ± 19.92 |
| 150 | | 81.95 ± 2.08 | 88.37 ± 6.37 | 89.183 ± 11.79 |
| 125 | | 76.01 ± 0.59 | 79.32 ± 2.28 | 77.16 ± 7.51 |
| 100 | | 61.52 ± 9.19 | | 69.61 ± 3.02 |
| 90 | | 58.41 ± 6.54 | | 63.46 ± 2.71 |
| 80 | | 45.86 ± 7.16 | | 60.74 ± 0.00 |
| 200 | | 43.23 ± 10.93 | 37.11 ± 11.58 | 39.09 ± 5.88 |
| 150 | | 46.74 ± 19.66 | 30.94 ± 5.51 | 32.38 ± 4.88 |
| 125 | | 36.76 ± 11.40 | 27.106 ± 6.26 | 32.14 ± 11.32 |
| 100 | | 28.15 ± 13.89 | | 20.41 ± 3.36 |
| 90 | | 18.23 ± 7.45 | | 17.15 ± 1.92 |
| 80 | | 13.63 ± 2.14 | | 13.71 ± 0.00 |
| 200 | −5.92 ± 3.19 | −3.44 ± 2.40 | −1.48 ± 1.38 | −8.14 ± 4.48 |
| 150 | −2.37 ± 0.83 | −0.98 ± 0.69 | −1.71 ± 2.13 | −5.24 ± 2.31 |
| 125 | −0.70 ± 0.73 | −3.36 ± 3.94 | −1.88 ± 1.79 | −7.06 ± 4.13 |
| 100 | −1.46 ± 1.23 | −4.97 ± 2.52 | | −5.22 ± 1.73 |
| 90 | −1.01 ± 1.25 | −4.39 ± 3.22 | | −6.07 ± 2.54 |
| 80 | −1.40 ± 2.05 | −1.69 ± 1.91 | | −2.81 ± 0.00 |
| 200 | 1.00 ± 0.00 | 0.97 ± 0.01 | 0.97 ± 0.07 | 0.99 ± 0.07 |
| 150 | 1.01 ± 0.02 | 0.98 ± 0.01 | 0.97 ± 0.06 | 0.99 ± 0.08 |
| 125 | 1.00 ± 0.01 | 0.96 ± 0.03 | 0.98 ± 0.07 | 0.99 ± 0.08 |
| 100 | 1.00 ± 0.01 | 0.96 ± 0.02 | | 0.99 ± 0.08 |
| 90 | 1.00 ± 0.00 | 0.97 ± 0.02 | | 0.98 ± 0.07 |
| 80 | 0.99 ± 0.01 | 0.95 ± 0.05 | | 1.04 ± 0.00 |

Effects of 4-AP on Cardiac Physiology: Treatment with 4-AP (7 mM), a transient outward potassium current (Iₜₒ) blocker, prolonged APD₈₀ (p<0.001) at all tested pacing rates compared to blebbistatin, as expected. 4-AP treatment also prevented pacing the hearts at pacing rates faster than 125 ms BCL. Additionally, at 150 ms BCL, 4-AP prolonged Ca²⁺ RT (p<0.024) and slowed CV_{T} (p=0.001) (FIG. 3A).

Effects of Verapamil on Cardiac Physiology: In contrast to the multiple effects of 4-AP, the effects of verapamil, an L-type calcium channel blocker (I_{CaL}) was specific to the upstroke of the calcium transient (FIG. 3A). Verapamil prolonged Ca²⁺ RT at all pacing rates tested as shown in FIG. 3B (p<0.001). This could have contributed to the increase in Vₘ RT by this drug (p=0.004).

Acute Modulation of Cardiac Physiology by No Flow Ischemia

A separate set of hearts was perfused with Control solution and a short episode of ischemia was induced by turning off the perfusion to the heart for a 5 min period followed by reperfusion. Ischemia modulated multiple parameters of cardiac physiology as shown in FIG. 4 while reperfusion restored all of them to pre-ischemic (baseline) values. Activation/intensity maps during baseline, ischemia (5 mins) and reperfusion (5 mins) are shown in FIG. 4A. All activation/intensity maps are from the same heart and all three maps in each column were generated from optical data that was simultaneously recorded. Time-dependent responses of the 7 tested parameters during ischemia and reperfusion are illustrated in the graphs in FIG. 4B and Table 2 while TPP graphs for ischemia and reperfusion demonstrating significant modulation of cardiac physiology during ischemia and restoration during reperfusion are shown in FIG. 4C. Significant modulation of each parameter in each heart was determined with respect to the baseline (pre-ischemic, t=0) value from that same heart. Since no changes in any of the measured parameters are expected over the short duration (10 mins) of this protocol without any external perturbations, baseline values serve as control.

TABLE 2
Modulation of cardiac physiology by Ischemia and Reperfusion.

| Time | Vₘ RT | CV_{T} | CV_{L} | | Ca²⁺ RT | Vₘ − Ca²⁺ | NADH |
|---|---|---|---|---|---|---|---|
| (mins) | (ms) | (m/s) | (m/s) | AR | (ms) | Delay (ms) | (Normalized) |
| 0 | 4.26 ± | 0.33 ± | 0.63 ± | 1.43 ± | 6.10 ± | −11.17 ± | 1.00 ± |
| | 0.37 | 0.04 | 0.07 | 0.74 | 1.31 | 8.24 | 0.00 |
| 1 | 4.85 ± | 0.30 ± | 0.57 ± | 1.89 ± | 5.62 ± | −13.09 ± | 1.02 ± |
| | 0.35 | 0.06 | 0.07 | 0.21 | 1.82 | 12.39 | 0.01 |
| 2 | 4.86 ± | 0.28 ± | 0.53 ± | 1.94 ± | 5.52 ± | −8.52 ± | 1.03 ± |
| | 0.99 | 0.05 | 006 | 0.37 | 1.20 | 7.73 | 0.02 |
| 3 | 5.23 ± | 0.25 ± | 0.47 ± | 1.91 ± | 6.30 ± | −5.83 ± | 1.03 ± |
| | 0.69 | 0.04 | 0.09 | 0.43 | 1.26 | 4.41 | 0.01 |
| 4 | 4.85 ± | 0.23 ± | 0.42 ± | 1.84 ± | 6.41 ± | −3.51 ± | 1.04 ± |
| | 0.67 | 0.05 | 0.09 | 0.14 | 1.50 | 4.69 | 0.01 |
| 5 | 5.45 ± | 0.19 ± | 0.36 ± | 1.39 ± | 6.43 ± | −2.31 ± | 1.04 ± |
| | 0.81 | 0.04 | 0.11 | 0.76 | 1.43 | 2.76 | 0.01 |
| 6 | 5.17 ± | 0.28 ± | 0.50 ± | 1.79 ± | 6.25 ± | −4.33 ± | 1.04 ± |
| | 0.94 | 0.08 | 0.12 | 0.33 | 0.73 | 1.90 | 0.03 |
| 7 | 5.11 ± | 0.33 ± | 0.54 ± | 1.65 ± | 6.07 ± | −4.17 ± | 1.03 ± |
| | 1.65 | 0.06 | 0.08 | 0.26 | 1.43 | 3.26 | 0.03 |
| 8 | 4.31 ± | 0.32 ± | 0.56 ± | 1.02 ± | 6.22 ± | −6.22 ± | 1.03 ± |
| | 0.44 | 0.05 | 0.04 | 0.84 | 1.13 | 3.36 | 0.03 |
| 9 | 4.18 ± | 0.34 ± | 0.63 ± | 1.81 ± | 6.05 ± | −8.42 ± | 1.02 ± |
| | 0.44 | 0.02 | 0.02 | 0.03 | 1.75 | 3.37 | 0.02 |
| 10 | 4.37 ± | 0.33 ± | 0.61 ± | 1.84 ± | 5.80 ± | −6.99 ± | 1.02 ± |
| | 0.49 | 0.00 | 0.00 | 0.06 | 1.82 | 3.25 | 0.03 |

As expected, the first parameter that was significantly altered during ischemia was NADH intensity. A quick increase in NADH levels was observed, as early as 1 min into ischemia (p=0.024). This was followed by changes in electrophysiology. Specifically, ischemia slowed CV_{T} at 3 mins (p=0.003) and then prolonged Vₘ RT (p=0.010) at 5 mins. Changes in AR and Ca²⁺ RT were not statistically significant during the 5 min ischemic protocol. Lastly, Vₘ-Ca²⁺ delay was also decreased by ischemia (p=0.016), possibly due to prolonged Vₘ upstroke but unaffected Ca²⁺ upstroke.

Reperfusion restored all tested parameters to pre-ischemic baseline values as quickly as 1 min after start of perfusion. Such a quick response is possibly due to the short duration of the preceding ischemia.

### DISCUSSION

Described herein are techniques for triple parametric optical mapping for simultaneous measurements of Vₘ, Ca²⁺ and NADH, which allow studying MECC. The significance of this methodology in drug testing and cardiac disease studies is demonstrated by performing triple-parametric optical mapping in mouse hearts during blebbistatin, 4-AP and verapamil treatments as well as during ischemia and reperfusion. As demonstrated above, while blebbistatin and 4-AP modulated multiple parameters of cardiac physiology, the effects of verapamil were focused to a single parameter. Specifically, verapamil treatment induced prolongation of Ca²⁺ RT which could be expected with an I_{CaL} blocker. On the other hand, blebbistatin prolonged Vₘ and Ca²⁺ RTs while 4-AP caused prolongation of APD, Ca²⁺ RT as well as slowing of CV_{T}. This methodology was also applied to investigate the acute effects of ischemia and reperfusion. While ischemia affected multiple parameters including increase in Vₘ RT and NADH as well decrease in CV_{T} and Vₘ—Ca²⁺ delay, reperfusion restored all these parameters to baseline values. By simultaneously measuring multiple aspects of cardiac function, it was determined that changes in the metabolic state precedes the electrophysiological modulation during ischemia. Thus, by implementing triple parametric optical mapping, unexpected off targets effects of drugs and sequence of modulation of cardiac physiology in disease were determined.

Effects of Blebbistatin on Cardiac Physiology

Blebbistatin is a selective inhibitor of myosin II isoforms found in skeletal muscles with little to no effect on other myosin isoforms. blebbistatin binds to the myosin-ADP-Pi complex and interferes with the phosphate release process, leaving the myosin detached from actin thereby arresting cellular contraction and preventing energy consumption by contraction thus reducing metabolic demand¹⁷. Blebbistatin is widely used as an electromechanical uncoupler to study cardiac physiology by optical methods which require arresting the heart to prevent motion-induced artifacts. Blebbistatin is more advantageous to previously used electromechanical uncouplers in that its effects on cardiac physiology are minimal. Blebbistatin does not alter calcium transient amplitude, rise time or decay as well as effective refractory period and ECG parameters. However, mixed reports on its effects on rabbit APD have been previously published with groups demonstrating that blebbistatin either does not alter APD or that it prolongs APD. Differences in methodologies including experimental conditions, poor perfusion, and motion correction algorithms applied could account for some of these differences in results. For example, blebbistatin applied to an ischemic preparation is likely to reverse ischemia induced APD shortening, appearing to prolong APD.

Although the effects of blebbistatin are well studied, these studies were mostly performed on rat or rabbit hearts and tested a concentration range of 0.1-10 mM which is less than recently reported concentrations used in mouse hearts. In the example disclosed herein, 15 mM blebbistatin was used to arrest heart motion during optical mapping and determine the effects of this concentration of blebbistatin on mouse cardiac physiology. Blebbistatin altered 2 of the 10 parameters measured at a “normal” pacing rate (150 ms BCL, 400 bpm) for ex vivo hearts. Upstroke rise time of action potentials and calcium transients were prolonged during blebbistatin treatment. Additionally, at slower heart rate (200 ms BCL), CV_{T} was faster and this correlated with reduced NADH autofluorescence intensity. Lower NADH values could correspond to increased ATP availability which could in turn modulate the ion channel and gap junction activity that affect cardiac conduction. This example illustrates how the use of triple parametric optical mapping can uncover such interrelated MECC that support cardiac physiology.

Blebbistatin is fluorescent with solvent-specific spectral properties which could interfere with the measurement of relative changes in NADH autofluorescence intensity between treatments. Blebbistatin dissolved in DMSO (solvent used in this example) has an excitation/emission peak of 420/560 nm and the majority of the emission is above 500 nm. The design of this triple-parametric optical mapping system which filters NADH optical signals using a 450±50 nm filter, thus avoids the addition of blebbistatin fluorescence in NADH signals. Furthermore, exposure of blebbistatin-perfused hearts to UV light, at intensities and durations required for NADH imaging, does not cause cytotoxicity or significant changes in its electromechanical uncoupling properties.

Effects of 4-AP on Cardiac Physiology

4-AP is a potent inhibitor of the Iₜₒ currents and is used in treatment of multiple sclerosis. Inhibition of Iₜₒ, a Phase 1 repolarizing current could cause prolongation of APD. In cardiac tissue, 4-AP has been demonstrated to have a biphasic effect where APD shortening is observed at lower concentrations but APD prolongation is induced at higher concentrations (>˜5 mM). This could be due to the inhibitory effects of 4-AP on other ion currents like I_{Kur} and hERG. In the presence of isoproterenol, 4-AP also promotes EADs and DADs in cardiomyocytes.

In this example, APD prolongation was observed by 7 mM 4-AP treatment, as expected, at all studied pacing rates. Additionally, this APD prolongation prevented 1:1 capture at pacing at rates faster than 125 ms BCL in mouse hearts. However, we report here that the effects of 4-AP on cardiac physiology extend beyond the expected blocking of the Iₜₒ current.

An inverse relationship between Phase 1 repolarization and calcium transient amplitude has been reported. Decrease in Phase 1 repolarization rate has been demonstrated to increase I_{CaL}, calcium transient amplitude and rise time. In this example, an increase was shown in the calcium transient rise time in hearts treated with 4-AP further supporting this relationship. However, the non-ratiometric calcium dyes used in this example did not allow the accurate quantification of calcium transient amplitude.

Lastly, it was demonstrated that 4-AP slows CV_{T} in mouse hearts. Although the effects of 4-AP on cardiac conduction has not been previously reported, it has been demonstrated to restore conduction in injured neurons. Although the effects of 4-AP were not tested in the context of injury, it was shown that 7 mM 4-AP reduces conduction velocity in mouse hearts. The dose dependence and underlying mechanism of this response will need further investigation.

Effects of Verapamil on Cardiac Physiology

Verapamil, an I_{CaL} blocker used to treat angina, hypertension, tachycardia and other cardiac diseases also has hERG channel blocking properties. It is probably due to its inhibitory effect on both potassium and calcium currents that the APD response to verapamil treatment has produced mixed results in previous studies. Verapamil-induced APD prolongation, shortening and no change have been previously reported. The varying dose-dependent effects of verapamil on I_{CaL} versus hERG current as well as differences in experimental models and tissues could explain some of these differing results. Verapamil has also been reported to have age-dependent effects on cardiac electrophysiology³⁸. In this example, no statistically significant changes was shown in APD in mouse hearts treated with 1 mM verapamil.

Furthermore, verapamil does not alter depolarization-related parameters. Verapamil has been reported to not alter the rate of depolarization in cells with sodium-dependent depolarization or alter conduction velocity. In line with these findings, no statistically significant changes were shown in Vₘ RT and CV in mouse hearts treated with verapamil.

The effects verapamil on calcium handling are many. Inhibition of I_{CaL} by verapamil has been shown to reduce the amplitude of calcium transients and contractility. It has also been demonstrated to suppress calcium transient alternans and reduce spontaneous calcium release. Prolonged Ca²⁺ RT is shown here in verapamil-treated mouse hearts. Increase in Ca²⁺ RT despite reduced calcium transient amplitude, could suggest significantly decreased I_{CaL} and calcium release from the sarcoplasmic reticulum. Lastly, it is shown that verapamil did not induce any significant changes in calcium reuptake as indicated by no changes in CaTD and Cat parameters.

Modulation of Cardiac Physiology by Ischemia/Reperfusion

Ischemia is a condition which is caused by the reduction or lack of blood supply to heart tissue. Ischemia modulates multiple parameters of cardiac physiology, including all three components of physiology measured in this study. Although the acute and chronic effects of ischemia are well-established, this example is the first to simultaneously assess cardiac electrical, calcium handling and metabolic functions to determine the complex sequence of MECC. Some of the well-known effects of acute ischemia include ATP reduction (NADH increase), APD shortening, Vₘ RT increase, CV slowing, calcium alternans and spontaneous calcium release.

Changes in NADH due to acute cardiac ischemia occur within 15 s and reperfusion can restore it to baseline within 60 s. In this study, we used a 5 min no flow ischemia model to measure the changes in cardiac physiology during acute ischemia and reperfusion. Ischemia increased NADH levels in the tissue at the earliest time point measured (1 min) and remained elevated throughout the ischemic period. This was the first of the ten parameters measured to be modulated suggesting that ATP depletion underlies most other physiological effects of ischemia. Next, at 3 mins of ischemia, CV_{T} slowing was observed. This was followed an increase in Vₘ RT. The effect of reduced ATP on the phosphorylation state of depolarizing sodium current and gap junctions could underlie these effects. It is also important to note that the effects of ischemia on Vₘ RT could be underestimated because the optical action potential recorded in each pixel is an average of multiple cardiomyocytes. Therefore, it is possible that Vₘ RT is increased sooner in the ischemic period than measured with this approach. Lastly, ischemia also reduced the Vₘ—Ca²⁺ delay possibly due to slower depolarization (increased Vₘ RT).

Reperfusion restored all measured parameters to pre-ischemic values within 1 min of restarting the perfusion to the heart. The short duration of the ischemic period could account for the immediate return to baseline conditions. Future studies aimed at determining the sequence of restoration of cardiac physiology during reperfusion could include prolonged ischemia periods or more frequent recordings during the reperfusion period.

The example disclosed herein demonstrates for the first time the application of triple-parametric optical mapping, which allows studying metabolism-excitation-contraction coupling in the heart. Here, methodology was applied for drug cardiotoxicity testing and to study the modulation of cardiac physiology during ischemia/reperfusion. Ten parameters of cardiac physiology were identified in the example related to electrical excitation, calcium handling and metabolism that give important information on the state of the heart. A ten-parameter panel (TPP) graph was developed, which can give a quick overview of the effects of drugs or diseases on the heart. Using this approach, the effects of blebbistatin, 4-AP, and verapamil on mouse cardiac physiology were determined. While blebbistatin and 4-AP altered multiple aspects of cardiac physiology, the effects of verapamil were limited to calcium transient upstroke as expected with a calcium channel blocker. This demonstrates that triple parametric optical mapping is a valuable tool to study cardiotoxicity of drugs in preclinical trials, particularly to identify off-target effects. Current drug testing is limited primarily to QT interval testing. This field could greatly benefit from a more comprehensive assessment of cardiac physiology as is the case with triple parametric optical mapping. Lastly, we also applied this methodology to determine the sequence of modulation of the multiple facets of cardiac physiology during acute ischemia. Simultaneously measuring the three facets of cardiac physiology identified that changes in metabolism during acute ischemia precede the effects on electrophysiology. The critical applications of this methodology demonstrate the need and the significance of triple parametric optical mapping.

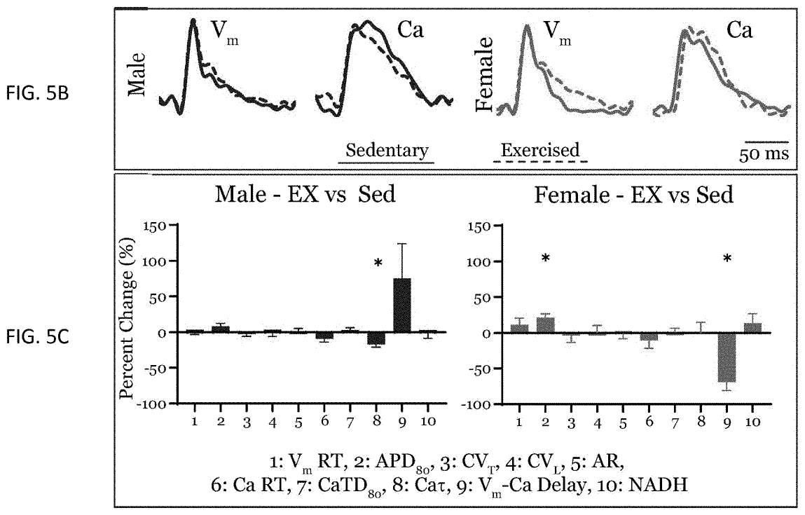

FIG. 5A is a map showing cardiac electrical, calcium handling and metabolic response to exercise. Simultaneously recorded NADH intensity maps and voltage and calcium activation maps are generated from triple-parametric optical mapping. All three maps in a given column are simultaneous recordings of the same field of view. FIG. 5B is a graph showing representative voltage and calcium traces from optical mapping of hearts in the four experimental groups. As shown, sedentary hearts are represented by the solid line, while exercised hearts are represented by dashed lines, for both males and females. FIG. 5C is a chart showing ten parameter panel illustrating changes in transmembrane potential, calcium handling and metabolism related parameters. Averages of percentage change in the exercised value versus the sedentary controls are reported for each parameter. As shown in the FIGS. 5A-5C, exercise modulates cardiac physiology in a sex-specific manner. Specifically, in males, calcium handling is disrupted where the reuptake of calcium back into the sarcoplasmic reticulum is shortened. On the other hand, in females, cardiac electrical function is disrupted by exercise whereby action potential duration and Vₘ-Ca delay is prolonged. These changes in cardiac MECC can make the heart more susceptible to arrhythmias.

The following references are hereby incorporated by reference. Ravens, U. Sex differences in cardiac electrophysiology. Canadian Journal of Physiology and Pharmacology (2018) doi:10.1139/cjpp-2018-0179. George, S. A., Lin, Z. & Efimov, I. R. Basic Principles of Cardiac Electrophysiology. in (2020). doi:10.1007/978-3-030-41967-7_1. Efimov, I., Nikolski, V. & Salama, G. Optical imaging of the heart. Circ. Res. 95, 21-33 (2004). George, S. A. & Efimov, I. R. Optocardiography: A review of its past, present, and future. Curr. Opin. Biomed. Eng. 9, 74-80 (2019). Efimov, I. R., Rendt, J. M. & Salama, G. Optical maps of Intracellular [Ca2+]i transients and Action Potentials from the Surface of Perfused Guinea Pig Hearts. Circulation (1994). Salama, G., Lombardi, R. & Elson, J. Maps of optical action potentials and NADH fluorescence in intact working hearts. Am. J. Physiol.—Hear. Circ. Physiol. (1987). Piccini, J. P. et al. Current challenges in the evaluation of cardiac safety during drug development: Translational medicine meets the Critical Path Initiative. American Heart Journal (2009) doi:10.1016/j.ahj.2009.06.007.

Janse, M. J., Kleber, A. G., Capucci, A., Coronel, R. & Wilms-Schopman, F. Electrophysiological basis for arrhythmias caused by acute ischemia. Role of the subendocardium. J. Mol. Cell. Cardiol. (1986) doi:10.1016/50022-2828(86)80898-7. Anyukhovsky, E. P. & Rosenshtraukh, L. V. Electrophysiological responses of canine atrial endocardium and epicardium to acetylcholine and 4-aminopyridine. Cardiovasc. Res. (1999) doi:10.1016/S0008-6363(99)00131-5. Dempsey, G. T. et al. Cardiotoxicity screening with simultaneous optogenetic pacing, voltage imaging and calcium imaging. J. Pharmacol. Toxicol. Methods (2016) doi:10.1016/j.vascn.2016.05.003. Ramanna, H. et al. Increased dispersion and shortened refractoriness caused by verapamil in chronic atrial fibrillation. J. Am. Coll. Cardiol. (2001) doi:10.1016/S0735-1097(01)01132-9. Cathey, B. et al. Open-Source Multiparametric Optocardiography. Sci. Rep. 9, (2019). Ratzlaff, E. H. & Grinvald, A. A tandem-lens epifluorescence macroscope: Hundred-fold brightness advantage for wide-field imaging. J. Neurosci. Methods (1991) doi:10.1016/0165-0270(91)90038-2.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention. All references cited herein are incorporated by reference in their entireties.

1. An optical mapping system comprising:
a plurality of light sources emitting different wavelengths of light to a cardiac tissue;
a first lens situated in a path of the light;
a first filter in the path of the light, wherein the first light filter outputs first filtered light;
a first sensor receiving the first filtered light and generating a first indicator of cardiac physiology;
a second filter in the path of the light, wherein the second light filter outputs second filtered light;
a second sensor receiving the second filtered light and generating a second indicator of cardiac physiology; and
a third light filter in the path of the light, wherein the third light filter outputs third filtered light;
a third sensor receiving the third filtered light and generating a third indicator of cardiac physiology;
wherein the first indicator of cardiac physiology, the second indicator of cardiac physiology, and the third indicator of cardiac physiology are mapped simultaneously.

2. The optical mapping system of claim 1, further comprising a second lens positioned between the first light filter and the first sensor.

3. The optical mapping system of claim 1, further comprising a third lens positioned between the second light filter and the second sensor.

4. The optical mapping system of claim 1, wherein the parametric mapping analyzes metabolism-excitation-contraction coupling in cardiac tissue.

5. The optical mapping system of claim 1, wherein the first, second, and third indicators of cardiac physiology are NADH, calcium, and voltage.

6. The optical mapping system of claim 1, wherein the first excitation light source has a wavelength of approximately 520 nm and the second excitation source has a wavelength of approximately 365 nm.

7. The optical mapping system of claim 1, wherein the system further generates one or more activation maps and one or more intensity maps from the first, second, and third indicators of cardiac physiology.

8. The optical mapping system of claim 1, wherein the system simultaneously measures up to ten physiological parameters during simultaneous mapping.

9. The optical mapping system of claim 8, wherein the up to ten parameters are related to repolarization and calcium reuptake.

10. The optical mapping system of claim 1, wherein the parametric mapping is used to indicate drugs effects or sequence of modulation of cardiac physiology in a disease of the cardiac tissue.

11. The optical mapping system of claim 1, wherein said first, second and third light filters each comprise a cube.

12. The optical mapping system of claim 1, wherein said first, second and third sensors each comprise a camera.

13. The optical mapping system of claim 1, further comprising a processing device mapping the first, second and third indicators of cardiac physiology.

14. A method of optical mapping comprising:
emitting different wavelengths of light from a plurality of light sources to a cardiac tissue;
installing a first lens in a path of the light;
installing a first light filter in the path of the light, wherein the first light filter outputs first filtered light to a first sensor generating a first indicator of cardiac physiology;
installing a second light filter in the path of the light, wherein the second light filter outputs second filtered light to a second sensor generating a second indicator of cardiac physiology; and
installing a third light filter in the path of the light, wherein the third light filter outputs third filtered light to a third sensor generating a third indicator of cardiac physiology;
wherein first indicator of cardiac physiology, the second indicator of cardiac physiology, and the third indicator of cardiac physiology are mapped simultaneously.

15. The method of optical mapping of claim 14, further comprising positioning a second lens between the first light filter and the first sensor.

16. The method of optical mapping of claim 14, further comprising positioning a third lens between the second light filter and the second sensor.

17. The method of optical mapping of claim 14, wherein the parametric mapping analyzes metabolism-excitation-contraction coupling in cardiac tissue.

18. The method of optical mapping of claim 14, wherein the first, second, and third indicators of cardiac physiology are NADH, calcium, and voltage.

19. The method of optical mapping of claim 14, wherein the first excitation light source has a wavelength of approximately 520 nm and the second excitation source has a wavelength of approximately 365 nm.

20. The method of optical mapping of claim 14, further comprising generating one or more activation maps and one or more intensity maps from the first, second, and third indicators of cardiac physiology.

21. The method of optical mapping of claim 14, further comprising simultaneously measuring up to ten physiological parameters during simultaneous mapping.

22. The method of optical mapping of claim 21, wherein the up to ten parameters are related to repolarization and calcium reuptake.

23. The method of optical mapping of claim 14, wherein the parametric mapping is used to indicate drugs effects or sequence of modulation of cardiac physiology in a disease of the cardiac tissue.
